(19) United States
(12) Patent Application Publication
(10) Pub. No.: US 2016/0139114 A1
(43) Pub. Date: May 19, 2016

(54) Rapid Test for Detecting Pathogen Material, in Particular in Order to Support the Diagnosis of Sepsis, and Kit and Device for Performing a Sepsis Test

(71) Applicant: GILUPI GMBH, Potsdam-Golm (DE)

(72) Inventors: Andreas Bollmann, Berlin (DE); Robert Niestroj-Pahl, Potsdam (DE); Klaus Lücke, Potsdam (DE); Solveigh Krusekopf, Berlin (DE)

(21) Appl. No.: 14/899,411

(22) PCT Filed: Jun. 23, 2014
(86) PCT No.: PCT/EP2014/063172

§ 371 Date: Dec. 17, 2015

(30) Foreign Application Priority Data
Jun. 21, 2013 (DE) 10 2013 211 850.3

(51) Int. Cl. G01N 33/543; G01N 33/569

(52) U.S. Cl. CPC G01N 33/54306; G01N 33/56911; G01N 2800/26

(57) ABSTRACT

A rapid test is provided for detecting pathogenic material, particularly for supporting the diagnosis of sepsis, as well as a kit and a device for the implementation of a sepsis test.

The present invention relates to a rapid test for detecting pathogenic material, particularly for supporting the diagnosis of sepsis, comprising the steps of bringing a sample in contact with at least one detection molecule under conditions that enable binding of a target molecule from the sample with the at least one detection molecule, as well as the further step of verifying whether a target molecule has bonded with the at least one detection molecule. The present invention further relates to a kit and a device for the implementation of a sepsis test.

Diagnostic criteria for sepsis and severe sepsis correspond to the ACCP/SCC Consensus Conference Criteria. This includes (1.) the detection of the infection by means of the microscopic detection or by means of clinical criteria; (2.) determination of the Severe Inflammatory Heat Response (SIRS) in which at least 2 of the following criteria have to be met:
fever (≧38° C.) or hypothermia (≦36° C.)
heart palpitations (tachycardia): heart frequency≧90/min.
excessive respiratory rate (tachypnea) or hyperventilation,
increased leukocyte level (leukocytosis) or
reduction of the leukocytes (leukopenia);

as well as (3.) detection of an acute organic dysfunction with at least one of the following criteria:
acute, pathological alteration of the brain (encephalopathy): restricted vigilance, disorientation, restlessness, delirium,
thrombocytopenia: decreased number of thrombocytes by more than 30% within 24 hours or number of thrombocytes≦100,000/mm³,
reduced blood oxygen level (arterial hypoxemia),
kidney failure (renal dysfunction),
hyperacidity of the blood (metabolic acidosis).

In the course of a sepsis, there is a frequent incidence of life-threatening disruptions of the vital functions and failure of one or several organs and eventually death of the patient. Intensive care medicine can bridge critical phases through temporary compensation or support of the organ functions. This, however, is only possible in cases where the existence of sepsis is detected in time. If there are already visible symptoms of sepsis such as an increased body temperature, heart rate or respiratory rate, it may already be too late for successful treatment.

As soon as visible symptoms arise and there is a suspicion of sepsis, there will be examinations to detect the pathogens for example in blood culture. However, the detection of the pathogen in blood culture takes at least 24, rather 48 hours. As this waiting time is too long in case of suspected sepsis, particularly intensive care patients are administered broad-spectrum antibiotics as a preventive and not targeted measure with the hope of them being effective with regard to the pathogen.

Further, there are biomarker examinations that are used to determine procalcitonin for early detection of sepsis. The determination of procalcitonin, however, can only be used to confirm an increased risk of the existence of a general infection. Such a test, in turn, does not replace a specific pathogen detection process and is not always able to substantiate a sepsis diagnosis either, because, depending on the pathogen, there can also be a sepsis in case of normal, unremarkable concentrations of procalcitonin.

Hence, the present invention is based on the purpose of providing a rapid test and a device that enable early identification of a pathogen for sepsis, detection of sepsis pathogens, determination of their pathogenic status and/or further characterization of the causative germ.

The present invention solves this problem through a rapid test for detecting pathogenic material comprising the steps:
bringing a sample into contact with at least one detection molecule that specifically binds a quorum sensing target molecule and/or a quorum sensing-associated target molecule under conditions that enable binding of the target molecule to the at least one detection molecule; and
verifying whether a target molecule has bonded to the at least one detection molecule.

The device according to the invention solves the problem by way of comprising at least one detection field with detection molecules that specifically bind a quorum sensing target molecule and/or a quorum sensing-associated target molecule. The kit according to the invention solves the problem by way of comprising at least one detection molecule that specifically binds a quorum sensing target molecule and/or a quorum sensing-associated target molecule, and a buffer, a washing reagent and/or a detection reagent to emit a measurable signal. Surprisingly, the inventors have found that an increased concentration of quorum sensing molecules and/or quorum sensing-associated molecules are a strong indicator of a sepsis risk and that the pathogens of sepsis and their pathogenic status can be determined by means of detecting quorum sensing target molecules and/or quorum sensing-associated target molecules. The invention enables a faster and more specific substantiation of a sepsis diagnosis than before and a further characterization of their pathogens. Detectable quorum sensing together with, for example, fever and hypotension is much more specific than fever, blood pressure and any inflammation parameter. This enables the practicing physicians at an intensive care unit and even at outpatient care units to take targeted measures for the treatment of sepsis, whereby the preventive administration of broad-spectrum antibiotics against all possible sepsis pathogens is avoided. The rapid test according to the invention and the process according to the invention enable in particular a detection of the infection by means of determining, inter alia, the gram status and the pathogenicity of the pathogen.

The device according to the invention is particularly suitable as a laboratory diagnostic tool close to the patient (point-of-care testing), i.e. to support diagnostic examinations that can be implemented in direct proximity to the patients, for example at the intensive care unit of the hospital, at outpatient care units or in emergency situations at a patient's apartment or in an ambulance, and not in a central laboratory.

A rapid test comprises examinations that can be carried out in direct proximity to the patients and whose result is available within a maximum of two hours, preferably in less than an hour, preferably within up to 15 minutes.

A specific binding in the sense of the present invention is a binding whose affinity is so high that it has an association constant (also called binding constant) of at least 10⁴ mol⁻¹, preferably of at least 10⁵ mol⁻¹ and particularly of more than 10⁶ mol⁻¹.

Quorum sensing target molecules are molecules that bacteria, yeasts and fungi communicate with. Quorum-sensing-associated target molecules are substances that are produced in an intensified way due to an increased concentration of quorum sensing molecules and/or that are altered in their function and/or structure compared to an environment without and/or with a low concentration of quorum sensing molecules.

Verification of whether a target molecule has bonded with the at least one detection molecule can be done in many different ways, for example by means of a colorimetric test, electrically or chemically, which will be addressed in even greater detail in the following. If the verification shows that a quorum sensing target molecule and/or a quorum sensing-associated target molecule has bonded with the at least one detection molecule, i.e. that it has been included in the sample, there is a case of sepsis.

The present invention can be further improved by means of a series of further developments that are each independent of one another and that are each advantageous in itself and that can be combined with each other in any way as described in the following.

According to a first embodiment, the kit according to the invention can comprise a device according to the invention and/or the sample can be brought in contact with a device according to the invention during a rapid test according to the invention.

According to another advantageous embodiment, the at least one detection molecule can be an antibody, a fragment of an antibody that is specifically binding, an antigen, a peptide, a protein, a nucleic acid, an aptamer, a ligand, a receptor, a hapten, an enzyme, an enzyme inhibitor, an enzyme substrate or a co-factor of an enzyme. The use of such detection molecules in the device/kit according to the invention and/or the rapid test according to the invention enables binding and detection of the target molecules with the required specific affinity. When using such detection molecules, it is also possible to verify by means of established assays, for example an immunoassay or an enzyme assay and/or an enzymatic analysis, whether a target molecule has bonded with the at least one detection molecule.

A blood sample, a urine sample, a sputum sample, a lymphatic fluid sample or another body fluid sample can be used a sample that is tested for the existence of sepsis by means of the rapid test according to the invention. The sample can be put onto the device according to the invention directly, i.e. without sample preparation, and/or can be brought directly in contact with at least one detection molecule when performing the rapid test according to the invention. Sample preparation methods such as filtration or precipitation of undesired sample components or disintegration of sample material are also possible according to the invention and can positively influence the result of the device and kit according to the invention and/or the rapid test according to the invention in the appropriate environment.

In another embodiment, the process according to the invention can comprise an intermediate step between the bringing into contact and the verifying of binding, in which non-binding sample material is removed. The detection molecules can be immobilized directly or indirectly through a linking molecule and/or a polymer matrix on a carrier for this purpose. For example, a test strip, a test stick, a micro-titer plate, a biochip, a micro-array substrate, but also a catheter, a cannula or a signal converter can be used as a carrier material. A device with immobilized detection molecules can, after the application of the sample, be treated with a washing solution and unbonded material can be washed off before the verification of whether a target molecule has bonded with the at least single detection molecule takes place. Test strips and test sticks have the advantage that the examination can run automatically after the application of the sample as the sample fluid is transported due to the capillary forces in the carrier strip and/or stick, for example of a nitrocellulose membrane, to the relevant reagents and detection fields. A micro-titer plate, a biochip and a micro-array substrate as carriers have the advantage that the device according to the invention is compact and that it can nonetheless be equipped with a plurality of detection fields. For example, each well of a micro-titer plate can form a separate detection field.

The device according to the invention can, regardless of its carrier and/or of the immobilization of the detection molecules on a carrier, comprise at least two detection fields, whereby the detection molecules specifically bind a different quorum sensing target molecule and/or quorum sensing-associated target molecule in each detection field. This way, the informative value of the rapid test, the kit and/or the device according to the invention can be improved as more than one target molecule, whose existence is an indicator for sepsis and/or that is typical for a specific pathogen, can be detected.

If a catheter or a cannula is used as a carrier of the device according to the invention, the test can be implemented particularly quickly, virtually in real time, because a blood sample is necessarily brought in contact with the detection molecules on the carrier during correct use of the cannula and/or of the catheter in this case.

Verification of a bond between the detection molecule and the target module can be facilitated if the detection molecules are immobilized on a signal converter, for example a signal converter of a biosensor. The signal line converts the binding between the detection molecule and the target molecule into a binding signal that can subsequently be transmitted through a wired or wireless signal line and read out by a relevant measurement unit. As a carrier, for example an electrochemical signal converter, an optical signal converter, an acoustic signal converter, an electric signal converter, a thermal signal converter, a piezoelectric signal converter or a biochemical signal converter can be used. Electrochemical and/or optical signal converters, that are robust and that enable a reliable conversion of the binding of a target molecule with a detection molecule into a measurable signal, are well suited. An electrochemical signal converter can convert the binding for example into a change of the resistance, the impedance or the electric current. An optical signal converter can express a change of the light refraction, as it occurs during surface plasmon resonance spectroscopy, as a binding signal. For example, histidine kinase can be used as a biochemical signal converter. Histidine kinases are enzymes that carry out an auto-phosphorylation process. Histidine kinases can be coupled with a detection molecule in a way that the histidin kinase will only be activated after binding of a target molecule with the coupled detection molecule. The activation triggers an auto-phosphorylation process in which ATP can be transformed into ADP. The ATP transformation, in turn, can be expressed as a binding signal and for example be visualized by way of colorimetry or converted into an electric binding signal through an electron transfer on a semiconductor.

According to another embodiment of a device according to the invention with a catheter as a carrier material, the device according to the invention can further comprise a signal converter that converts the binding of a target molecule on the at least one detection field equipped with detection molecules into a binding signal, as well as a signal line for the transmission of the binding signal. This way it will be possible to detect the target molecule that is sepsis-specific and/or typical for a pathogen type in real time if the catheter of this embodiment is placed correctly, for example as a heart catheter or urinary catheter, as it is equipped with all components of a conventional biosensor, which enable the conversion transmission of a signal of the binding.

As already mentioned above, the detection molecules can be immobilized on a carrier directly or indirectly through a linking molecule (also called linker) and/or a polymer matrix. Thanks to the versatile characteristics, a polymer matrix is well suited for the modification of the carrier surface, and the plurality of different polymers and possibilities to modify these polymers allow for the selection of a polymer matrix specifically for the respective purpose of the device according to the invention. For example, a polymer matrix that has functional groups through which the detection molecules can be tied directly is well suited. Further advantageous features of a polymer matrix are listed in the following:
the polymer matrix is designed as a continuous polymer layer. Therefore, the whole surface of the carrier can be covered or shielded by the polymer matrix. The thickness of the polymer layer is preferably in the range of 0.1 to 10 μm, preferably in the range of 0.5 to 5 μm, particularly preferably in the range of 1 to 2 μm.
The polymer matrix has a three-dimensional, preferably filamentous and/or porous structure. The polymer matrix has a carbonic, branched molecule structure. These structures are very suitable for binding the detection molecules and increase the effective binding surface. In the area of the boundary layer on a surface that is covered with this molecule structure, the circulation of a sample fluid is slowed down significantly. Therefore, the enrichment of the ligands is favored. Possible preferred embodiments of the structured functional surface are a functional surface with ridges, indentations and/or branches and/or a functional surface that comprises at least partially a spiral-shaped, screw-shaped, winding, wave-shaped, helical, filamentous, brush-shaped, comb-shaped, net-shaped, porous, sponge-shaped structure.
The polymer matrix is preferably effectively connected to the carrier, for example the catheter or the signal converter, via functional groups, preferably through a chemical bond, particularly preferably through a covalent bond.
The polymer matrix is a hydrogel.
The polymer matrix comprises saturated groups of atoms and covalently bonded detection receptors in order to avoid undesired interactions with blood components and the binding of unspecific cells and molecules.
The polymer matrix is cross-linked.
The polymer matrix comprises and/or forms the detection field. The polymer matrix can be loaded directly with the detection molecules.
The polymer matrix prevents binding of unspecific cells or interactions with body fluids.
The polymer matrix can transfer electrons.
The polymer matrix can consist of modifiable substances that can structurally change when a target molecule binds with a detection molecule and that induce optical and/or electronic signals in this way.

It is also possible to immobilize the detection molecules through linking molecules on the respective detection field that is provided for this purpose.

According to another preferred embodiment of the rapid test according to the invention, a detection reagent that emits a measurable binding signal can be added for verifying the binding process. In case of binding, the detection reagent can trigger for example a color reaction or a chemical reaction that is measurable, for instance, through a pH change. Molecules that specifically bind the compound of detection molecule and target molecule are preferably used. A detection reagent can for example bind a second epitope of the target molecule that is not occupied by the detection molecule. Options for detection reagents are antibodies that are equipped with a marker, proteins, aptamers or ligands. The marker can emit a measurable binding signal directly, for example in form of a fluorescence signal or a color scheme, or indirectly produce the bonding signal, for example create a coloring agent or free electrons as part of an enzymatic substrate transformation, which can be measured by way of colorimetry or by means of a semiconductor.

According to a further embodiment, the device according to the invention can comprise a measurement unit to detect at least one target molecule. Preferably, the device according to the invention comprises a measurement unit to detect the bond between the detection molecule and the target molecule in each detection field. The measurement unit allows for a quantitative detection and indicates, in case of a realization according to the invention with more than one type of detection molecule, which target molecules are existing. An automation of the rapid test and the handling of a device according to the invention can be improved for example by a digital meter that indicates the binding. To detect the target molecule not only qualitatively, but also to quantitatively determine the quantity of the target molecule, the measurement unit can preferably be used not only to detect but also be designed to quantify the binding between the detection and the target molecule in at least one, preferably in each detection field. The rapid test according to the invention can also provide for a quantification of the measurable binding signal. The quantification of the quantity of the target molecule in the sample has the advantage that it can be used to draw conclusions about the progression of the sepsis. The strength of the binding signal, that can correlate with the quantity of the target molecule that has bonded with the at least one detection molecule, can for example be used for the quantification. Further, the quantification process can run semi-quantitatively in a way that a binding signal is only emitted visibly if a specific threshold value of a concentration of the target molecule in the sample is exceeded.

But not only the progression of the sepsis can be detected with the device according to the invention, the kit and the rapid test. According to a further embodiment, the sepsis pathogen, preferably the gram status of the sepsis pathogen, can be characterized as a function of the target molecule that binds specifically with the detection molecule. Thereby, the condition that quorum sensing target molecules and/or quorum sensing-associated target molecules are in particular only produced by gram-negative pathogens and/or gram-positive pathogens is beneficial. For example, homoserine lactone is a gram-negative-specific quorum sensing target molecule. Quorum sensing oligopeptides, that are summarized as auto-inductor peptides and that include for instance furanosyl borate diester, are specific for gram-positive pathogens. Exemplary homoserine lactones are contained in the group of the N-acyl-homoserine lactones (HSL) 1 to 4 (HSL1: N-(11-carboxy-3-oxoundecanoyl)-L-homoserine lactone; HSL2: N-(5-carboxypentanoyl)-L-homoserine lactone; HLS3: N-(11-carboxy-3-hydroxyundecanoyl)-L-homoserine lactone; HSL4: N-(9-carboxynonanoyl)-L-homoserine lactone). In a rapid test with a detection molecule for specifically binding with respectively one of the HSL 1-4 molecules and/or with a device that is equipped with four detection fields with detection molecules against HSL 1 to 4, conclusions can be drawn even on a specific pathogen type as a function of the binding pattern.

The same applies for detection molecules that specifically bind a quorum sensing-associated target molecule. Surprisingly, it has become apparent in this context that the enzyme activities of beta-galactosidase, beta-hexosaminidase and arylsulphatase A are increased and that the enzyme activity of paraxonase 1 (also paraoxonase 1) is reduced as a direct consequence of an increased concentration of quorum sensing molecules. The enzyme activity of beta-galactosidase, beta-hexosaminidase and arylsulphatase A is increased at least 4-fold, preferably at least 10-fold, compared to the normal value in case of sepsis. In case of sepsis, the paraxonase 1 activity is reduced by at least 33%, preferably by approximately 50% or more, compared to the activity without sepsis.

The detection of such a quorum sensing-associated enzyme can for example be implemented in the rapid test according to the invention in a way that the target molecule is a quorum sensing-associated enzyme, especially beta-galactosidase, beta-hexosaminidase, arylsulphatase A and/or paraxonase 1 whose enzyme activity is determined. If, during the determination of the enzyme activity, it turns out that the latter is increased and/or reduced, this shall be interpreted as the proof of sepsis. According to another embodiment, the detection molecules can specifically bind with one of the quorum sensing target molecules homoserine lactone, preferably N-acyl-homoserine lactone 1, N-acyl-homoserine lactone 2, N-acyl-homoserine lactone 3, N-acyl-homoserine lactone 4, an auto-inductor peptide, preferably a furanosyl borate diester, or with one of the quorum sensing-associated target molecules beta-galactosidase, beta-hexosaminidase, arylsulphatase A, paraxonase 1 in the at least one detection field. Thereby, any potential combination of the abovementioned quorum sensing target molecules and/or quorum sensing-associated target molecules is possible and disclosed herein, e.g. HSL 1 and beta-galactosidase, even if it is not explicitly stated in the application text.

In the following, the invention will be described in an exemplary way by means of drawings. The combinations of features explained with reference to the drawings, however, can be changed according to the above descriptions. Hence, individual features of the device according to the invention can for example be omitted if these features do not offer a substantial advantage in a defined application. Similarly, one of the features described above can be added if the benefit in connection with this feature is advantageous for the respective application.

The drawings show:

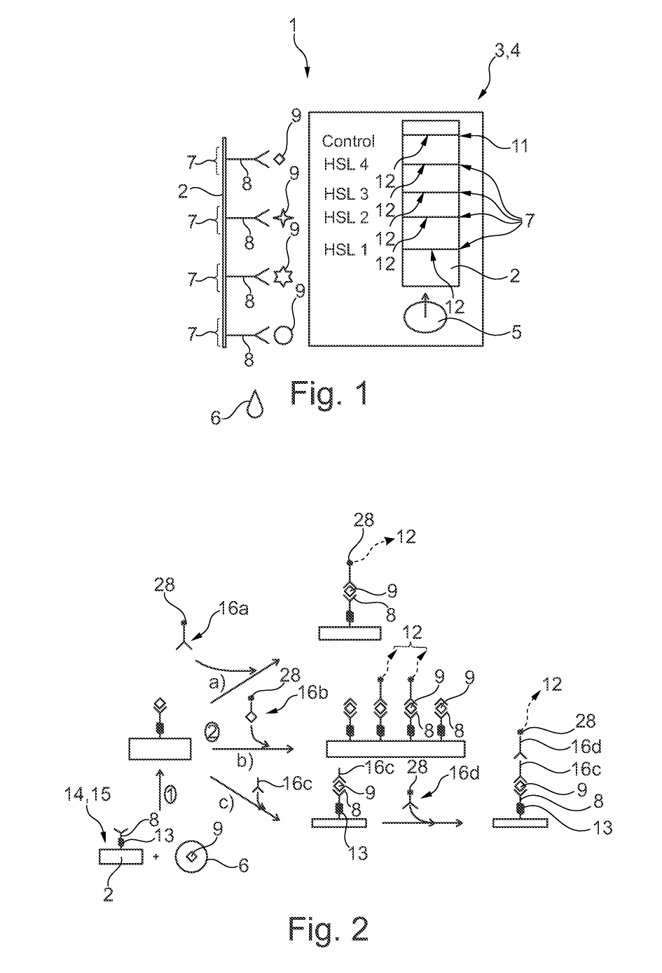

FIG. 1 a device according to the invention in a schematic display according to a first embodiment;

FIG. 2 a schematic display of different variants of a process according to the invention;

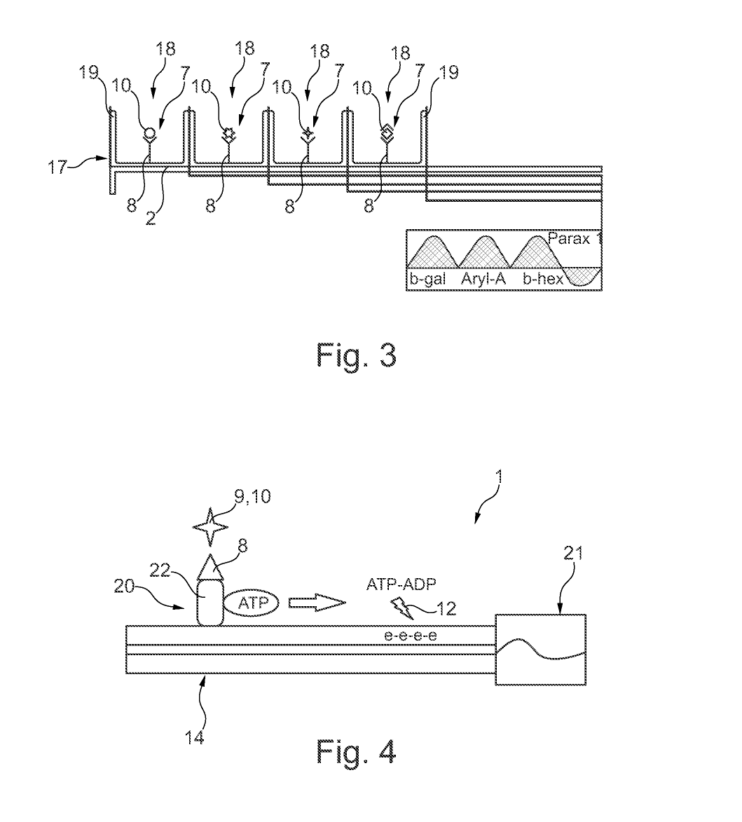

FIG. 3 a schematic display of a device according to the invention in accordance with a second embodiment;

FIG. 4 a schematic display of a device according to the invention in accordance with a third embodiment;

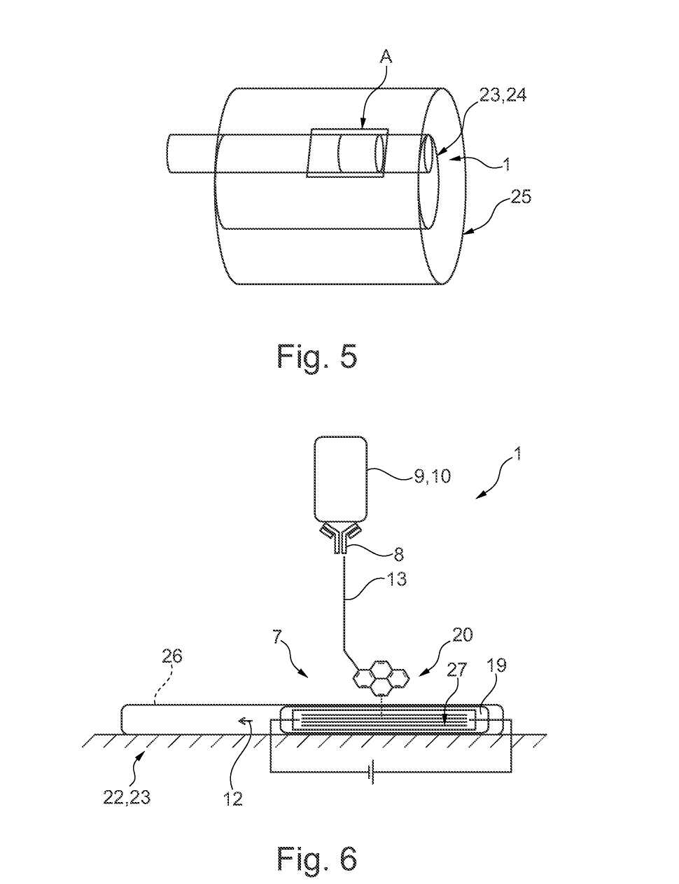

FIG. 5 a schematic perspective display of a fourth embodiment of the device according to the invention; and

FIG. 6 a schematic display of section A from FIG. 5.

The device 1 according to the invention and its individual components will be described in detail with reference to the attached drawings by means of exemplary embodiments in the following. At the same time, the kit according to the invention as well as the rapid test for the detection of pathogenic material of a sepsis will be explained with reference to the embodiments of the device 1 according to the invention that are shown in the figures.

The device 1 according to the invention, the kit as well as the rapid test according to the invention enable the detection of pathogenic material to support the diagnosis of a sepsis in a fast and simple way and in a reliable manner.

A first embodiment of the device 1 according to the invention will be explained in greater detail with reference to FIG. 1 in the following. In the embodiment shown in FIG. 1, the device 1 according to the invention has a carrier 2 that is designed in the style of a test strip 3 and/or test stick 4. Such test strips 3 and/or test sticks 4 are particularly suitable as rapid tests for laboratory diagnostics close to the patients. The device 1 comprises an application field 5 on which a sample 6, that is shown in the illustrated embodiment as a blood sample displayed by means of a drop of blood, is applied. The blood sample is transported due to capillary forces along the flow direction, which is illustrated as an arrow in FIG. 1, in the carrier 2, for example a nitrocellulose membrane. During transportation, the sample 6 passes at first through four detection fields 7 and subsequently through a control field 11 [“control”].

Each control field 7 is equipped with detection molecules 8 that specifically bind a quorum sensing target molecule and/or a quorum sensing-associated target module 10.

Antibodies are arranged as special detection molecules 8 in the four detection fields 7 in the displayed embodiment. In the embodiment illustrated in FIG. 1, the detection molecules 8 specifically bind the quorum sensing target molecules 9, i.e. N-acyl-homoserine lactone 1 (HSL 1: illustrated by a circle), N-acyl-homoserine lactone 2 (HSL 2: illustrated by a six-pointed star), N-acyl-homoserine lactone 3 (HSL 3: illustrated by a four-pointed star) and N-acyl-homoserine lactone 4 (HSL 4: illustrated by a rhombus).

If the sample 6 is brought in contact with the detection molecules 8 in the respective detection field 7, the respective target molecule 9 will specifically bind with the antibody 8 directed against it. In the illustrated embodiment of the device 1 according to the invention that is designed as a test strip 3 and/or test stick 4, the detection molecules 8 are immobilized directly on the carrier 2 so that the quorum sensing target molecules 9 binding the detection molecules will be held back in the respective detection field 7 if the sample 6 has passed a test field 7 along its flow direction.

The embodiment shown in FIG. 1 therefore comprises a device 1 according to the invention with four detection fields 7, whereby the detection molecules 8 in each detection field 7 specifically bind a different quorum sensing target molecule 9, i.e. HSL 1 to 4.

The control field 11 can have an element 8 (not shown) that indicates that the applied sample 6 has passed all detection fields 7 from the application field 5 in a flow direction and flowed into the control field 11. An antibody that is directed against a component that always exists in blood, for example a globulin- or album-directed antibody, can for example be immobilized in the control field 11. Alternatively, the control field can be designed in a way that it indicates only the wetting with sample fluid in a visible way by means of emitting a measurable binding signal 12.

In the illustrated embodiment of a test strip 3 and/or test stick 4, an optical binding signal 12, i.e. a color strip, is created as soon as a quorum sensing target molecule 9 specifically binds the respective detection molecule 8 in a test field 7 and/or when the applied sample reaches the control field that becomes visible as a colored strip.

The ways in which such a binding signal 12 can be generated are explained in greater detail in the following with reference to the exemplary embodiments of the rapid test according to the invention and to FIG. 2.

In the rapid test for detecting a sepsis according to the invention, a sample 6, for example a blood sample, urine sample, sputum sample, lymphatic fluid sample or another body fluid sample that is to be checked for the existence of a sepsis, is at first brought in contact with at least one detection molecule 8 that specifically binds a quorum sensing target molecule 9 from the sample 6 under conditions that enable binding of the target molecule 9 with the detection molecule 8. In the progression scheme shown in FIG. 2 that starts in the bottom left corner, the detection molecule 8 is illustrated in an exemplary way as an antibody that is immobilized through a linking molecule 13 (also called linker) on a carrier 2, for example on a biochip 14 or a micro-array substrate 15. After establishing the contact, the rapid test according to the invention includes the verification whether the target molecule 8 has bonded with the at least single detection molecule 9.

FIG. 2 displays three possible variants of verifying the binding of a target molecule 9 with a detection molecule 8 in an exemplary way: the direct proof with a detection reagent 16a (alternative 2a), the competitive proof by means of a detection reagent 16b (alternative 2b) as well as a so-called sandwich assay (alternative 2c).

For the direct proof, a further antibody is added as a detection reagent 16a that emits a measurable binding signal 12. The further antibody that is used as a detection reagent 16a is equipped with a marker 28 and able to specifically bind the target molecule 9 that is bound to the detection molecule 8. The binding of the detection reagent 16a on the target molecule 9 is indicated by a binding signal 12 generated by the marker 28 of the detection reagent 16a. The binding signal can for example be a color signal if the marker 28 is a fluorescent compound or an enzyme that catalyzes a color reaction.

In the competitive assay, an analyte that competes with the target molecule 9 for the binding place on the detection molecule 8 is added as a detection reagent 16a. The analyte that forms the detection reagent 16b is also equipped with a marker 28 that is designed in a way as to be able to emit a binding signal. The competitive assay is advantageous as it can be used not only to qualitatively detect that the target molecule 9 is contained in the sample 6, but also to quantitatively determine the quantity of target molecule 9 in the sample. The more target molecules 9 are contained in the sample 6, the more target molecules 9 remain bonded on the detection molecule 8 after adding the detection reagent 16b. This means that, in case of a high concentration of the target molecule 9 in the sample 6, only few molecules of the detection reagent 16b will bind with the detection molecules 8 and hence a weak binding signal will be emitted. If, in contrast, there are only few or no target molecules 9 in the sample 6, almost all and/or all detection molecules 8 will be occupied by the detection reagent 16b and a correspondingly strong binding signal 12 will be emitted.

Another option to verify the binding of a target molecule 9 with a detection molecule 8 is the so-called sandwich assay that is also used in conventional ELISA (enzyme-linked immunosorbent assay) processes. In a first detection step, a secondary antibody 16c that is able to bind specifically with the target molecule 9 that is bonded on the detection molecule 8 is thereby used. Subsequently, the actual detection reagent 16d is added in a second step, i.e. a further antibody 16d that is equipped with a marker 28 and that binds the secondary antibody 16c specifically. The resulting sandwich structure in which the marker 28 of the detection reagent 16d emits a measurable binding signal 12 is displayed at the bottom right in FIG. 2.

The reagents 16a-16d that lead to the indication of the measurable binding signal 12 can be placed, in case of the test strip 3 and/or test stick 4 from FIG. 1, for example in the flow direction ahead of the corresponding detection field 7 in the carrier material in a way that the sample 6 carries the reagents 16a-16d along and transports them into the detection fields 7.

FIG. 3 shows a schematic display of a device 1 according to the invention 1 in accordance with a second embodiment. The following part will only address the differences in relation to the device 1 of the previous figures. The same reference signs will be used for elements whose function and/or structure is identical to elements of the previous figures.

In device 1 that is shown in FIG. 3, a micro-titer plate 17 is used as a carrier 2. The individual wells 18 of the plate 17 are equipped with respectively one detection field 7.

Each detection field 7 is equipped with a detection molecule 8 that binds a specific quorum sensing-associated target molecule 10. Each detection field 7 is equipped with another detection molecule 8 so that a different quorum sensing-associated target molecule 10 can be detected in each detection field 7. The antibodies 8 that constitute the detection molecules 8 in the device 1 from FIG. 3 bind, when contemplated from left to right, the following quorum sensing-associated target molecules 10: beta-galactosidase (illustrated with a circle), beta-hexosaminidase (illustrated with a six-pointed star), arylsulphatase A (illustrated with a four-pointed star) and paraxonase 1 (illustrated with a rhomboid).

In contrast to the previous embodiment, the detection molecules 8 in the detection fields 7 of the device 1 shown in FIG. 3 are linked to the carrier 2 neither directly nor by means of a linker, but are coupled to a polymer matrix 19 that is applied to the internal wall of the wells 18. By means of an appropriate choice of the polymer matrix 19, for example if this polymer matrix has functional groups, a large number of detection molecules 8 can be immobilized in the respective wells 18. Further, there is a plurality of polymers that are biocompatible and that prevent unspecific binding of target molecules 9, 10.

In the embodiment shown in FIG. 3, the target molecules 10 represent quorum sensing-associated enzymes whose activities are changed in case of a sepsis compared to healthy patients as a direct consequence of an increased concentration of quorum sensing molecules. The change of the quorum sensing-associated target enzymes 10 is indicated schematically by the waves at the bottom right in FIG. 3. Hence, the activity of the quorum sensing-associated target molecules beta-galactosidase, beta-haxosaminidase and arylsulphatase A is increased and the enzyme activity of paraxonase 1 is reduced in case of sepsis. For example, it can be determined whether there is a changed enzyme activity of the quorum sensing-associated target molecules 10 for example by means of determining the concentration of the respective quorum sensing-associated target enzyme 10 in the sample or measurement of the respective enzyme activity. A defined threshold activity and/or threshold concentration can be used, for example, as a reference or a comparable sample of a healthy patient who is not infected with sepsis can be measured.

In the device 1 shown in FIG. 3, the respective detection molecules 8 in the corresponding well 18 do not have to be immobilized on the carrier 2 and/or the polymer matrix 19. The detection molecules can also be placed in the well 18 in a stable dosage form, for example in a crystallized state, in a way that they will dissolve in a functional form if the sample 6 is added. Further, it is possible to put the respective detection molecules 8 with the sample 6 separately into the respective indentation and to subsequently implement the rapid test according to the invention after the sample 6 and the detection molecules 8 have been brought in contact in the well 18.

Subsequently, a third embodiment of a device 1 according to the invention will be addressed in greater detail with reference to FIG. 4.

In FIG. 4, the detection molecule 8 is immobilized on the signal converter 20. The signal converter 20, in turn, is connected to a biochip 14 that is equipped with a semiconductor. As will be explained in even greater detail in the following, the semiconductor absorbs free electrons when a target molecule 9, 10 is binding the detection molecule 8 and emits an electric current as a binding signal 12 that can be displayed and quantified by a measurement unit 21, i.e. a digital measurement unit in the illustrated embodiment.

A histidine kinase 22 is used as a signal converter 20 in the embodiment of FIG. 4. The histidine kinase 20 is connected at one place with the semiconductor plate with the biochip 14 and on the other hand coupled with the detection molecule. The coupling process forms a complex of the detection molecule 8 and the histidine kinase 22. As soon as the target molecule 9, 10 accumulates at the detection molecule 8, the histidine kinase 22 of the complex is activated and triggers a hydrolysis reaction of ATP that is bonded in the histidine kinase. During the hydrolysis, a phosphate residue is obtained from the ATP, ADP is formed and energy is released. The energy that is released during the ATP hydrolysis can be transferred potentiometrically to the semiconductor by means of an energy carrier, which can in turn be indicated as a binding signal 12 and detected by means of the measurement unit 21. In the device shown in FIG. 4, the strength of the electric current that correlates with the reaction energy can not only detect the qualitative binding of quorum sensing target molecules 9 and/or quorum sensing-associated target molecule 10 on the detection molecule 8, but also provide quantitative information about the quantity of the target molecule 9, 10 in the sample.

In the following, a device 1 according to the invention in accordance with a fourth embodiment will be explained with reference to FIGS. 5 and 6. For elements whose function and/or structure is identical or similar to elements of the preceding figures, the same reference signs will be used in FIGS. 5 and 6. In the fourth embodiment, a catheter 23 and/or a cannula 24 is the carrier 2 of the device 1 according to the invention.

FIG. 5 shows how the catheter 23 and/or the cannula 24 is placed in a lumen 25 that is shown in an exemplary way and that can, for example, be a blood vessel. The drawings illustrate the indications of size of the catheter 1, the lumen 3 as well as the device 1 in a merely schematic way and not true to scale.

The device 1 from FIG. 5, whose section A is schematically displayed in detail in FIG. 6, comprises an electric conductor as a signal conductor 26, a signal converter 20 that is coated with a polymer 19 and a detection field 7 that comprises antibodies as detection molecules 8. The antibody 13 is not bonded to the polymer 19 directly but coupled through a linking molecule 13. Linkers are small molecules that have for example two equal (homobifunctional) or two different (heterobifunctional) functional groups. Likewise, the length of the linkers is relevant for the function. Zero-length cross-linkers are used for a bond of two molecules without a spacer.

Particularly in case of complex molecules such as enzymes or antibodies, the use of linkers can have a favorable effect on the biological activity of the immobilized structure. Through the linker, the active center or the active domain of the molecule is brought further away from the stem structure onto which the molecule is immobilized. This reduces the risk of inactivation due to the immobilization. Another possibility consists of choosing the linker in a way that it only binds to a defined structure in the target molecule and therefore keeps the active area of the molecule intact. For the coupling of an IgG antibody to carboxyl groups in the polymer, the zero-length cross-linker EDC (1-ethyl-3[3-dimethylaminopropyl]carbodiimide hydrochloride) can be used, which catalyzes the formation of a peptide bond between a primary amino group in the antibody and a carboxyl group of the polymer.

In the embodiment shown in FIG. 6, the signal converter 20 comprises an electrode arrangement 27 that measures a change of the resistance. The resistance change is triggered by the binding process of the target molecules 9, 10 and a related structural change of the linking molecule 13, emitted as a binding signal 12 by the signal converter 20 and transported to the outside through the signal conductor 26.

Binding of the target molecule 9, 10 can for example leave behind an electric signal if the binding partner is an electroactive substance and changes its conductivity, for instance through a mass increase due to the binding. In case of a rapid test for quorum sensing-associated enzymes as target molecules 10, the signal emission can occur potentiometrically as the charge carriers are set free due to the enzymatic transformation and indicate the electric current change as a measurement value. In optical terms, the binding can be detected or proven by way of colorimetry or as the binding changes the light reflection/refraction features, which can be detected by light particle sensors.

### REFERENCE SIGNS

1 Device
2 Carrier
3 Test strip
4 Test stick
5 Application field
6 Sample
7 Detection field
8 Detection molecule
9 Quorum sensing target molecule
10 Quorum sensing-associated target molecule
11 Control field
12 Binding signal
13 Linker
14 Biochip
15 Micro-array substrate
16a, 16b, 16d Detection reagent
16c Secondary antibody
17 Micro-titer plate
18 well
19 Polymer matrix
20 Signal converter
21 Measurement unit
22 Histidine kinase
23 Catheter
24 Cannula
25 Lumen
26 Signal conductor
27 Electrode arrangement
28 Marker
A Section in FIG. 5

1. A device, comprising at least one detection field with detection molecules that specifically bind a quorum sensing target molecule or a quorum sensing-associated target molecule.

2. The device of claim 1, in which the at least one detection molecule is an antibody, a fragment of an antibody that is specifically binding, an antigen, a peptide, a protein, a nucleic acid, an aptamer, a ligand, a receptor, a hapten, an enzyme, an enzyme inhibitor, an enzyme substrate or a co-factor of an enzyme.

3. (canceled)

4. The device of claim 1, comprising at least two detection fields, whereby the detection molecules specifically bind a different quorum sensing target molecule and/or a different quorum sensing-associated target molecule in each detection field.

5. The device of claim 1, in which the detection molecules are immobilized on a carrier directly or indirectly through a linking molecule and/or a polymer matrix.

6. The device of claim 5, in which the carrier is a test strip, a test stick, a micro-titer plate, a biochip, a micro-array substrate, a catheter, a cannula or a signal converter.

7. The device of claim 1, wherein at least one detection field comprises a measurement unit for detection of and/or for quantification of binding between the detection molecule and the target molecule in at least one, preferably in each detection field.

8. A kit for the implementation of a sepsis test, comprising at least one detection molecule that specifically binds a quorum sensing target molecule and/or a quorum sensing-associated target molecule,
and at least one of a buffer, a washing reagent, or a detection reagent to emit a measurable signal.

9. A method of detecting pathogen in a sample, comprising:
bringing a sample into contact with at least one detection molecule that specifically binds a quorum sensing target molecule and/or a quorum sensing-associated target molecule under conditions that enable binding of the target molecule with the at least one detection molecule; and
verifying whether a target molecule has bonded with the at least one detection molecule.

10. The method of claim 9, wherein the sample is brought in contact with a device comprising at least one detection field with detection molecules that specifically bind either a quorum sensing target molecule or a quorum sensing-associated target molecule.

11. The method of claim 9, wherein the sample is a blood sample, a urine sample, a sputum sample, or a lymphatic fluid sample.

12. The method of claim 9, further comprising adding a detection reagent that emits a measurable binding signal, for verifying the binding process.

13. The method of claim 12, further comprising quantifying the measurable binding signal.

14. The method of claim 9, in which the target molecule is a quorum sensing-associated enzyme, and the method further comprises determining activity of the quorum sensing-associated enzyme.

15. The method of claim 9, further comprising characterizing the pathogen by determining the gram status of the pathogen as a function of the target molecule that specifically binds with a detection molecule.

16. The device of claim 1, in which the detection molecules in the at least one detection field specifically bind with:
a. a quorum sensing-associated target molecule selected from the group consisting of a homoserine lactone, and an auto-inductor peptide, or
b. a quorum sensing-association molecule selected from the group consisting of: beta-galactosidase, beta-hexosaminidase, arylsulphatase A, and paraoxonase 1.

17. The device of claim 16, in which:
a. the detection molecules in the at least one detection field specifically binds with a homoserine lactone selected from the group consisting of N-acyl-homoserine lactone 1, N-acyl-homoserine lactone 2, N-acyl-homoserine lactone 3, and N-acyl-homoserine lactone 4, and/or
b. the detection molecules in the at least one detection field specifically binds with a furanosyl borate diester.

18. The method of claim 10, in which the detection molecules in the at least one detection field specifically bind with:
a. a quorum sensing-associated target molecule selected from the group consisting of a homoserine lactone, and an auto-inductor peptide, or
b. a quorum sensing-association molecule selected from the group consisting of beta-galactosidase, beta-hexosaminidase, arylsulphatase A, and paraoxonase 1.

19. The method of claim 9, wherein the sample is a bodily fluid sample.

20. The method of claim 9, in which the pathogen is a sepsis pathogen and the method supports a diagnosis of sepsis in a patient.

21. The method of claim 14, wherein the quorum sensing-associated enzyme is selected from the group consisting of beta-galactosidase, beta-hexosaminidase, arylsulphatase A and paraoxonase 1.
